(19) United States
(12) Patent Application Publication
(10) Pub. No.: US 2012/0238491 A1
(43) Pub. Date: Sep. 20, 2012

(54) IMMUNOLOGICALLY ACTIVE LIPOPEPTIDES AND METHODS FOR USE THEREOF

(75) Inventor: James Douglas Thacker, Doylestown, PA (US)

(73) Assignee: THERIMUNEX PHARMACEUTICALS, INC., Doylestown, PA (US)

(21) Appl. No.: 13/263,755

(22) PCT Filed: Apr. 8, 2010
(86) PCT No.: PCT/US10/30433

§ 371 Date: Jun. 11, 2012

Related U.S. Application Data
(60) Provisional application No. 61/168,283, filed on Apr. 10, 2009.
(60) Provisional application No. 61/228,170, filed on Jul. 24, 2009.
(60) Provisional application No. 61/239,251, filed on Sep. 2, 2009.

(51) Int. Cl. A61K 38/10; A61P 31/04; A61P 31/12; C07K 17/02; C12N 5/0775

(52) U.S. Cl. 514/2.4; 530/326; 435/366; 514/3.7

(57) ABSTRACT

The present invention relates to the isolation and structure elucidation of 1-peptidyl-2-arachidonoyl-3-stearoyl glyceride (pDAG) as an active chemical entity in the caprine serum fraction, and the discovery of the innate immune modulating activity of pDAG as an endogenous damage associated molecular pattern

### CROSS REFERENCE TO RELATED APPLICATIONS

This application is a non-provisional application, which is incorporated by reference herein and claims priority of U.S. Provisional Application No. 61/168,283, filed Apr. 10, 2009, U.S. Provisional Application No. 61/228,170, filed Jul. 24, 2009, and U.S. Provisional Application No. 61/239,251, filed Sep. 2, 2009.

### BACKGROUND OF THE INVENTION

1. Field of the Invention

Generally, the present invention relates to the field of immunologically active peptides, and more specifically to 1 peptidyl-2-arachidonoyl-3-stearoyl glyceride, which has been discovered to have immune modulating activity making it suitable for the treatment and mitigation of infectious diseases and other pathological conditions.

2. Background

The search for new medically useful natural products is a well accepted process that frequently results in lead molecules that are further developed as pharmaceutical agents. Despite the fact that these new agents rarely become marketable drug products, a large percentage of drugs currently in use are derived from natural products, such as morphine, paclitaxol, and quinine Biological sources such as bacterial and mold cultures, plant extracts, marine, and aquatic life forms have been traditional places to look for these unique natural products. Rarely however, has mammalian serum been used as a biological source to mine for new natural products with a potential for medicinal use. Hamm and associates, in Hamm, D; Willeford, K. O; White, G; and Reed, S. M. Equine Vet. J. 2002, 34,71-75, reported a serum fraction derived from the goat (Capra hircus) that was effective as an adjunctive therapy with standard antibiotics for treatment of suppurative lower respiratory disease in horses. They reported that 86% of horses treated with the serum fraction, in addition to standard antibiotics, recovered within three weeks, whereas only 10% of horses treated with antibiotic alone recovered. To the best of our present knowledge there have been no further reports in the literature that describe the agent or agents responsible for this effect.

### SUMMARY OF THE INVENTION

In the development of the present invention a series of experiments were carried out to isolate the agent or agents responsible for the effect described by Hamm and associates, Hamm, D; Willeford, K. O; White, G; and Reed, S. M. Equine Vet. J. 2002, 34, 71-75. The experiments were undertaken to isolate a component of mammalian serum that is effective at mediating an innate immune response. This resulted in the isolation and structure elucidation of 1-peptidyl-2-arachidonoyl-3-stearoyl glyceride (hereinafter, pDAG) as an active chemical entity in the caprine serum fraction, and the discovery of the innate immune modulating activity of pDAG as an endogenous damage associated molecular pattern. pDAG produced in accordance with the present invention has the structural formula represented below:

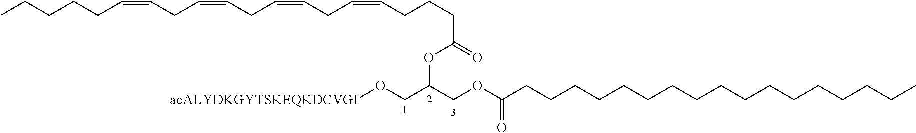

In addition, in accordance with another feature of the present invention, a dose response relationship for inflammatory cytokine and chemokine production and release from human fibroblasts incubated with nanomolar concentrations of pDAG is provided. Moreover, the apparent membrane transport role of the diacylglycerol moiety in pDAG has been discovered, with nanomolar quantities of the transfected N-acetyl peptide moiety of pDAG also inducing inflammatory cytokine and chemokine production and release. The apparent EC₉₉ for pDAG is 3 ng/mL.

### BRIEF DESCRIPTION OF THE DRAWINGS

FIG. 1. Human fibroblast IL-6 mRNA expression induced by synthetic and natural compound pDAG. Human fibroblasts were incubated for 24 h with either synthetic pDAG (5 ng/mL) or the purified natural pDAG (1:100 dilution). The result from synthetic pDAG at 5 ng/mL is statistically equivalent to the natural pDAG at a 1:100 dilution (p>0.05) and both natural pDAG and synthetic pDAG induced nominally 30% more IL-6 mRNA expression relative to the untreated control (p<0.01). Data is normalized to -actin mRNA expression.

FIG. 2. IL-8 dose response to synthetic pDAG. IL-8 mRNA expression (top) and secreted IL-8 protein (bottom) from human fibroblasts after treatment with increasing concentrations of synthetic pDAG show a negative dose response to mRNA expression and a positive dose response to secreted protein. Statistical significance relative to the control is indicated as *(p<0.05) or **(p<0.001).

FIG. 3. Comparison of natural and synthetic pDAG-induced MCP-1 and MIP-1 mRNA expression by human fibroblasts. (Top) MCP-mRNA expression in human fibroblasts is approximately equivalent (3-fold increase) for natural pDAG (1:100 dilution) and synthetic pDAG (3 ng/mL). Data are normalized to -actin expression. (Bottom) MIP-1 mRNA expression by natural pDAG (1:100 dilution) and synthetic pDAG (3 ng/mL) are equivalently increased in human fibroblasts ˜60% relative to the -actin control. Statistical significance relative to the control is indicated as *(p=0.01), **(p=0.001), and ***(p=0.003).

FIG. 4. Comparison of the amino acid sequences of the peptide in pDAG to TRPC-1. Sequence homology of the peptide in pDAG with the internal amino acid sequence from bovine, human, and murine TRPC-1. The internal serine is substituted with proline in bovine TRPC-1. The N-terminal X in pDAG is N-acetylalanine and the C-terminal X is pDAG-stearoyl-2-arachidonoyl-sn-glycerol.

FIG. 5. Cytokine mRNA induced by 10 ng/mL (˜3 nM) of the transfected peptide from pDAG or diacylglycerol. IL-1 , IL-6, IL-8, IL-18, and IL-33 mRNA were substantially increased over untreated control fibroblasts or fibroblasts treated with lipofectamine alone. The panel in the lower right corner shows the effect on mRNA expression for IL-1 , IL-33, and IL-18 in fibroblasts incubated with 12.5 ng/mL (˜30 nM) diacylglycerol alone as compared to untreated control fibroblasts. Data are normalized to -actin mRNA expression.

### DETAILED DESCRIPTION OF THE INVENTION

Through a series of dialysis and chromatographic separations, native compound pDAG was isolated from normal goat serum as a tryptophan complex. We estimated the initial concentration of pDAG in serum to be 50 ng/mL. No compound pDAG was detectable in normal plasma. Fractions generated during the purification procedures were chromatographically analyzed and assayed for their ability to induce IL-8 mRNA expression and IL-8 secretion by human fibroblasts. A preparative C₈ reversed phase (RP) HPLC fraction containing the biologically active compound pDAG/tryptophan complex was used for spectrometric analysis.

Functional group characterization using two dimensional H NMR(400 MHz) showed multiple amide signals (4-5 ppm) indicating a peptide, an aliphatic lipid prosthetic group (0.8-1.2 ppm), and signals between 8-9 ppm indicating aromaticity (data not shown). There was no signal in the ³²P NMR spectrum indicating the absence of a phosphorus atom in the natural product (data not shown). Therefore we subjected a sample of the RP-HPLC-purified natural compound pDAG/tryptophan complex to Edman degradation. The amino acid sequence identified was XLYDKGYTSKEQKD(CVGI . . . ) where the N-terminal amino acid indicated by X was either a non-traditional amino acid or was masked by a prosthetic group. The sequence of the C-terminal amino acids that were identified had a degree of uncertainty due to the low molar abundance of peptide remaining after the previous 14 analysis cycles.

A quantity of pDAG (as a tryptophan complex) was collected by preparative scale RP-HPLC and analyzed by liquid chromatography/mass spectrometry (LC/MS) and LC/tandem mass spectrometry. The major component in the LC/MS analysis eluted at 1.4 min., exhibited strong absorbance at 218 and 280 nm, and had a MW of 204 (Supporting Information, Figure S2, including the positive-ion electrospray ESIMS of the major component). LC/MS-MS analysis of the m/z 205 ion ([M+H]⁺) was consistent with the structure for tryptophan. Further evidence was obtained by comparing the LC/MS-MS spectra of the unknown MW 204 component with an authentic standard of tryptophan and confirmed the presence of tryptophan at an estimated molar ratio of 200:1 relative to pDAG.

Additional automated LC/MS-MS experiments were performed to investigate the presence of low-level peptides. The ion trap mass spectrometer was configured to acquire MS-MS spectra of all components above a relatively low signal threshold. One of the peptides was found to have a MW of 1689 Da and was comprised of a series of fragment ions (m/z 261.09, 340.27, 564.00, and 845.45) that was consistent with the partial sequence identified by Edman degradation. The ion at m/z 845.45 was determined to be a M²⁺ ion after deconvolution and observing 0.5 amu separation of the isotopemers. The fragment ion series from ESIMS-MS analysis of the m/z 845.45 ion (Table 1, below) is consistent with the sequence acetylALYDKGYTSKEQKD, which includes the partial sequence (X₁)LYDKGYTSKEQKDCVGI(X₂) identified by Edman degradation where X₁ and X₂ are either derivatized amino acids or non-amino acid prosthetic groups.

TABLE 1
ESIMS-MS Ion Fragments and Assignments for the
N-terminal Peptide Fragment of pDAG

| fragment ion, m/z | ion type | assignment |
|---|---|---|
| 845.45 | y²-ion | acALYDKGYTSKEQKD(OH) |
| 787.9 | y²-ion | LYDKGYTSKEQKD(OH) |
| 778.9 | b²-ion | acALYDKGYTSKEQK |
| (100%) | | |
| 714.8 | b²-ion | acALYDKGYTSKEQ |
| 649.8 | y²-ion | DKGYTSKEQKD(OH) |
| 592.3 | y²-ion | KGYTSKEQKD(OH) |

MALDITOFMS analysis was used before and after hydrolysis and derivatization to identify the C-terminal sequence of amino acids in the peptide and to identify the C-terminal prosthetic group as 1-stearoyl-2-arachidonoyl glycerol (DAG). No molecular ion corresponding to the intact molecule pDAG was observed; however, four major ion fragments were detected in the positive-ion spectrum without added matrix (tryptophan served as a matrix in this experiment). The high mass fragment (m/z 1282.71) was interpreted as arising from the loss of neutral NH₃ from the N-terminal fragment ion [acetylALYDKGYTSKE]⁻. The low mass fragment (m/z 1133.30) was consistent with the y-ion containing the C-terminal diacylglycerol (“DAG”) [DCVGI-(DAG)]⁺ and the base peak (m/z 1208.47) was consistent with a tryptophan adduct of the internal z-ion fragment [KEQKDCVGI]W⁻. The corresponding y-ion (+15 amu) was also observed at m/z 1223.61. The y-ion at m/z 1207.53 was assigned to the C-terminal fragment [TSKEQKDCVGI]⁺.

Co-crystallization of the isolated product with dihydroxybenzoic acid and re-analysis by MALDITOF mass spectrometry gave an expanded series of ion fragments that accounted for the entire structure of the bioactive molecule. However, we failed to observe a molecular ion corresponding to the intact parent molecule. The ion fragments and their structural assignments are presented in Table 2, below.

TABLE 2
MALDITOFMS Ion Fragments for pDAG

| fragment | ion | |
|---|---|---|
| ion, m/z | type | assignment |
| 645.76 | y-ion | DAG |
| 761.45 | y-ion | I(DAG) |
| 1170.48 | b-ion | acALYDKGYTSK |
| 1620.02 | x-ion | KEQKDCVGI(DAG) |
| 2016.70 | a-ion | acALYDKGYTSKEQKDCVGI |

It was observed that the serum-derived compound pDAG was especially labile to hydrolysis under acidic and basic conditions which may account for the difficulties in obtaining a molecular ion under either ESI or MALDI ionization conditions. The C₈ RP-HPLC peaks eluting between 4.83 and 9.36 min. in a non-purified fraction containing compound pDAG are hydrolysis products of compound pDAG. Therefore, the classical degradation approach to natural product structure elucidation was used. The purified compound pDAG (as a tryptophan complex) was subjected to mild acid hydrolysis, in situ N-terminal sulfonation, and analysis of the hydrolysis products to identify peptide fragments that would account for the entire structure of pDAG. The PSD MALDITOF analysis of the N-terminal sulfonated peptide fragments that were generated after mild acid hydrolysis of pDAG showed multiple y-ions consistent with previously observed peptide fragments and accounted for the complete structure of pDAG (see Table 3, below).

TABLE 3
MALDITOFMS of Hydrolysis Products from pDAG

| fragment ion, m/z | assignment |
|---|---|
| 961.27 | [TSKEQKDC]Na⁺ |
| 945.29 | [GYTSKEQK]Na⁺ |
| 927.27 | [acALYDKGYT]⁺ |
| (a-ion) | |
| 857.23 | [TSKEQKD]Na⁺ |
| 848.27 | [KEQKDCV]⁺ |
| 825.26 | [acALYDKGY]⁺ |
| (a-ion) | |
| 758.20 | [I-DAG]⁺ |
| 756.19 | [SKEQKD]Na⁺ |
| 734.18 | [SKEQKD +H₂O]⁺ |
| 656.15 | [KDCVGI]Na⁺ |
| 646.14 | [KEQKD +H]⁺ |
| 645.16 | [DAG]⁺ |
| 634.16 | [KDCVGI]⁺ |
| 568.20 | [QKDCV −28]Na⁺ |
| 545.10 | [QKDCV −28]⁺ |

Edman degradation sequence analysis, ESI-MSⁿ analysis of the purified natural product, and mild acid hydrolysis followed by MALDITOFMS of the hydrolysis products allowed for a putative chemical structure to be assigned to pDAG as acetylALYDKGYTPKEQKDCVGI-DAG, where DAG is 1-stearoyl-2-arachidonoyl-sn-glycerol prosthetic group (X₂ in the Edman degradation sequence analysis) esterified to the C-terminal isoleucine and N-acetylalanine is the derivatized amino acid (X₁) that could not be identified by Edman degradation sequence analysis.

Synthesis of pDAG and the peptide moiety were undertaken to establish the chemical structure of pDAG and to begin to elucidate its biological function. The chemical equivalence of synthetic pDAG and natural pDAG was established by ESIMS analysis.

To determine the bioequivalence between the natural product and synthetic pDAG, normal human fibroblasts were exposed to pDAG and IL-6, IL-8, MCP-1 and MIPlα a expression were measured. IL-6 has both pro-inflammatory and anti-inflammatory actions and is secreted by a wide variety of cell types in response to pathogen-associated molecular patterns (PAMPs). See, for example, Janeway, C. A. Cold Spring Harb. Symp. Quant. Biol. 1989, 54, 1-13 and Akira, T.; Takeda, K.; Kaisho, S. Nat. Immunol. 2001, 2, 675-680. IL-6 is one of the most important mediators of the acute phase response. See, e.g., Kishimoto, T.; Akira, S., Narazaki, M., Taga, T. Blood 1995, 86, 1243-1254 and Heinrich, P. C.; Behrmann, I.; Haan, S.; Hermanns, H. M.; Müller-Newen G.; Schaper, F. Biochem. J. 2003, 374, 1-20. IL-8 (CXCL8) is a chemoattractant protein of the CXC chemokine family and is also an important mediator of the inflammatory response. See Baggiolini M.; Clark-Lewis, I. FEBS Lett. 1992, 307, 97-101. Secreted by a wide variety of cell types, IL-8 functions to recruit neutrophils to phagocytose pathogens and other foreign antigens. MCP-1 (CCL2) is a member of the CC chemokine family that is secreted in response to cell injury or pathogen infection thus recruiting monocytes, memory T cells, and dendritic cells to the site of injury or infection. See Can, M. W.; Roth, S. J.; Luther, E.; Rose, S. S.; and Springer, T. A. Proc. Natl. Acad. Sci. U.S.A. 1994, 91, 3652-3656. MIP-1 (CCL3) or macrophage inflammatory protein activates neutrophils and stimulates the production and release of other pro-inflammatory cytokines such as IL-1, IL-6, and TNF-, Hsieh, C. H.; Frink, M.; Hsieh, Y. C.; Kan, W. H.; Hsu, J. T.; Schwacha, M. G.; Choudhry, M. A.; Chaudry, I. H. J. Immunol. 2008, 181, 2806-2812.

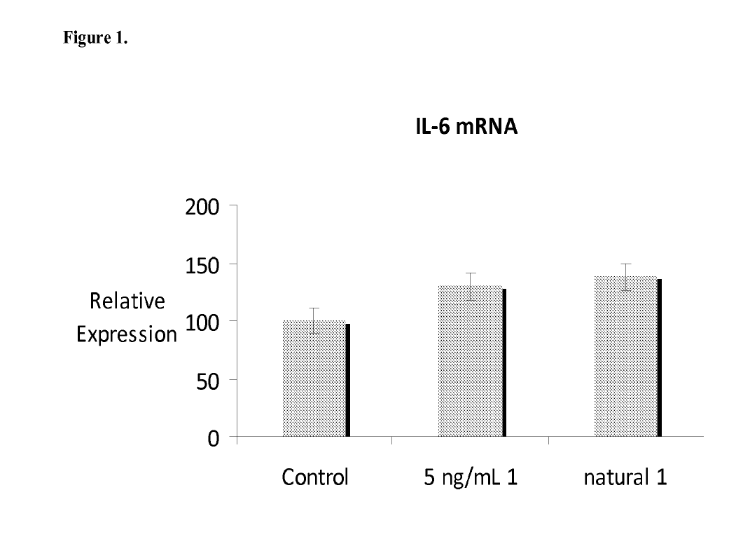

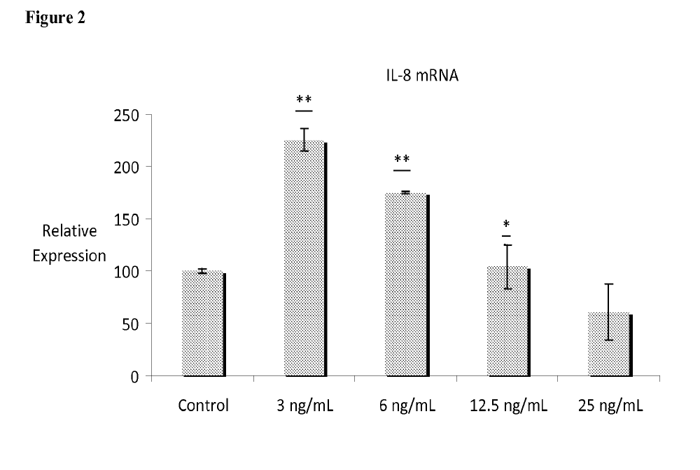

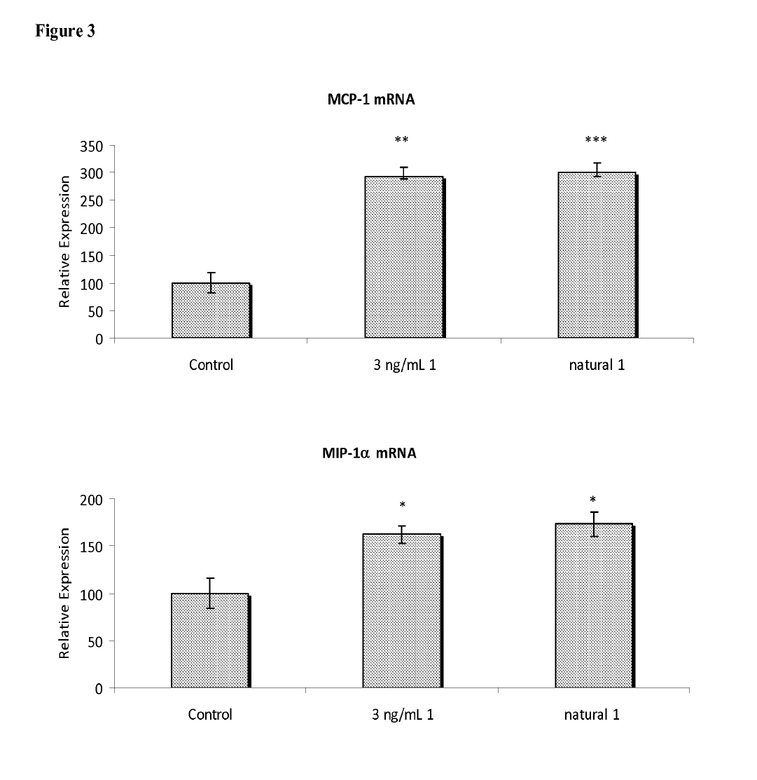

Fibroblasts were treated with various concentrations of synthetic pDAG for 24 hours; the media were reserved for ELISA assays of IL-6 and IL-8, and mRNA was extracted from the cells to measure all four cytokines IL-6 mRNA was elevated 20% in response to synthetic pDAG and 40% with natural pDAG relative to a -actin control (p<0.05). It was found that 5 ng/mL of synthetic pDAG was approximately equivalent to the natural product diluted 1:100 (FIG. 1). Compound pDAG induced IL-8 expression by about 5-fold compared to untreated fibroblasts, in dose-response studies (FIG. 2). The negative dose response relationship with respect to mRNA expression suggests possible cell toxicity at the higher concentrations of pDAG. Secreted IL-8 measured by ELISA showed a positive linear dose response with increasing concentrations of synthetic pDAG from 0.3 to 3 ng/mL, where the latter concentration appears to give a maximal effect (EC₉₉). The MCP-1 and MIP-1 mRNA expression in human fibroblasts incubated with approximately equivalent amounts of natural pDAG (1:100 dilution) and synthetic pDAG (3 ng/mL). Both synthetic pDAG and the natural product induced a 3-fold increase in MCP-1 mRNA relative to the -actin control and increased MIP-1α mRNA expression relative to -actin by 15% (FIG. 3).

The foregoing experimental results were used to establish the bioequivalence of the natural and the synthetic products. Further, a possible maximum effective concentration (EC₉₉) for pDAG at 3 ng/mL (nominally 1 nM) was established. These results indicate that compound pDAG is effective at a “cytokine-like” concentration.

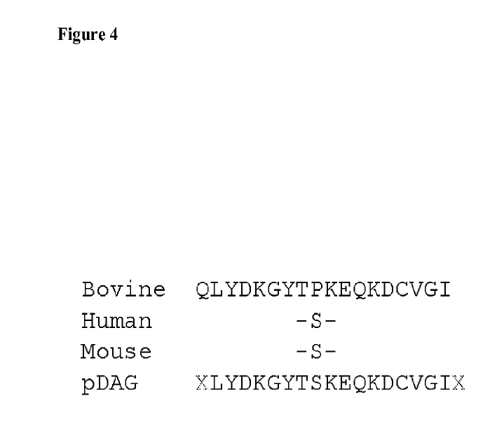

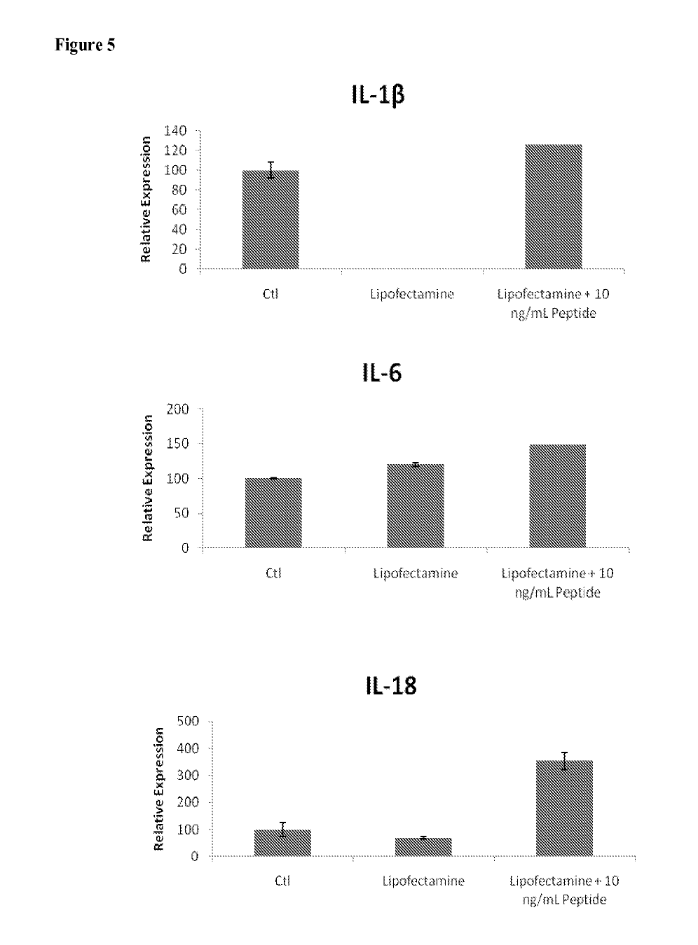

Given the amino acid sequence for the peptide in pDAG, the non-redundant sequence database for homologous sequences was searched using the NCBI BLAST search tool BLASTP n http://blast.ncbi.nhm.nih.gov/Blast.cgi,. See Altschul, S. F.; Madden, T. L.; Schaffer, A. A.; Zhang, J.; Zhang, Z.; Miller, W.; Lipman, D. J. Nucleic Acid Res. 1997, 25, 3389-3402. The peptide showed identical sequence homology to amino acids 558-574 in the transient receptor potential channel-related protein 1 (TRPC-1). No significant homology with other known proteins or peptides was observed. A comparison of the amino acid sequence at positions 557-574 from bovine, mouse, and human TRPC-1 with the peptide sequence in pDAG is presented in FIG. 4 where X represents the N- and C-terminal prosthetic groups of pDAG. The TRPC family of proteins belongs to the TRP super family of non-voltage-gated cation channel proteins and seven TRPC family members have been described in mammals. TRPC-1 is the human homolog to Drosophila TRP first discovered by Wes and associates. See Wes, P. D.; Chevesich, J.; Jeromin, A.; Rosenberg, C.; Stetten, G.; Montell, C. Proc. Nat. Acad. Sci. USA 1995, 92, 9652-9656. Members of the TRPC family, including TRPC-1, appear to be conserved within the animal kingdom and the trp gene family is expressed in a wide variety of tissues and cells types including immune cells. See Hardie, R. C. J. Physiol. 2007, 578, 9-24. The membrane topology of TRPC-1 suggests that the amino acids comprising the peptide are extracellular, in the region between the sixth membrane spanning unit and the pore-forming seventh membrane spanning unit.¹³ Sequence homology of this region of TRPC-1 with the other members of the TRPC family is relatively low (˜30%) as compared to ˜80% for the N-terminal sequence. See Wes, et al., supra.

The trpc1 gene is located on human chromosome 3 at position q22-q24. Interestingly, trpc1 is expressed as multiple unique transcripts and splice variants, and has multiple loci for NF B binding. See Paria, B. C.; Malik, A. B.; Kwiatek, A. M.; Rahman, A.; Mays, M. J.; Ghosh, S.; Tiruppathi, C. J. Biol. Chem. 2003, 278, 37195-37203. This raises the possibility that the region coding for the peptide is a separate reading frame for transcription. Examination of the trpc1 gene and its various splice variants revealed that no stop codon corresponding to the C-terminal isoleucine was present. Therefore, it is unlikely that the peptide in pDAG is transcribed from trpc1 as a separate reading frame. TRPC-1 is assembled in the endoplasmic reticulum (ER) and we hypothesize that ER embedded TRPC-1 is the source of this peptide perhaps in response to cell injury or infection.

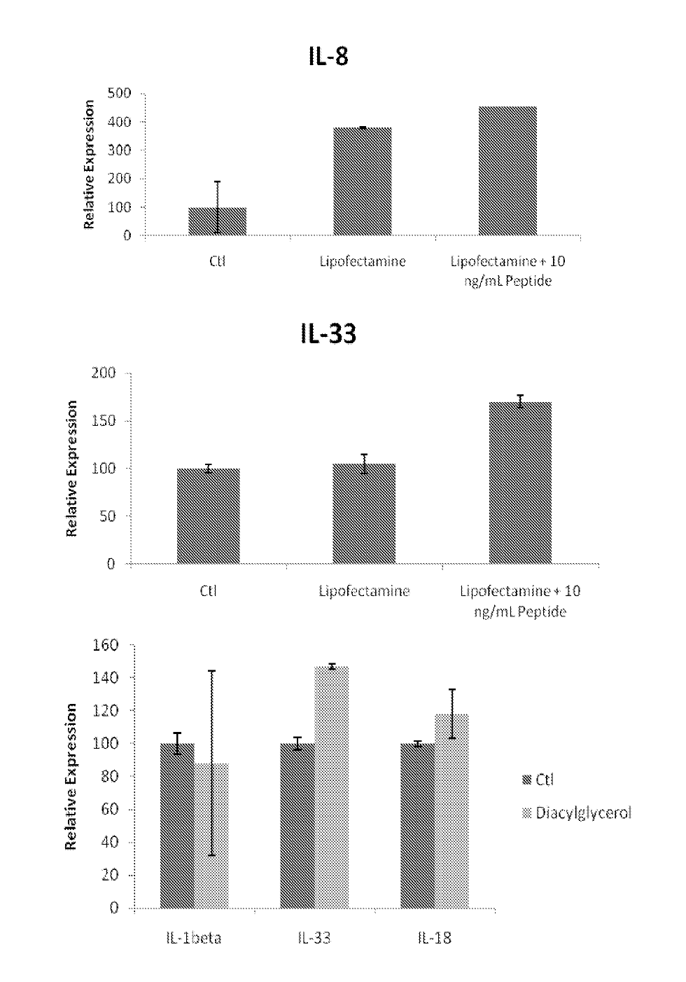

We observed that the peptide moiety in compound pDAG did not elicit comparable responses in fibroblast stimulation until it was 1,000-fold more concentrated (3 M). Therefore we speculated that the diacylglycerol moiety enabled the peptide to cross the cell membrane. To test this hypothesis and to ascertain the biological role of the diacylglycerol moiety in compound pDAG, if any, the peptide moiety transfected into human fibroblasts or diacylglycerol alone were evaluated by measuring the expression of mRNA (IL-1, IL-6, IL-8, IL-18, and IL-33). We found that the peptide/lipofectamine complex enabled the peptide (˜10 nM) to cross the cell membrane and elicit similar effects as compound pDAG (FIG. 5). Diacylglycerol could stimulate mRNA expression of IL-33 and IL-18, but not IL-1 in fibroblasts. However, diacylglycerol required a 3-fold higher molar concentration (˜30 nM) to achieve the same percent increase in IL-33 mRNA expression (40-50%) as the transfected peptide (FIG. 5). Moreover, diacylglycerol only increased the IL-18 mRNA expression by 20% as compared to the transfected peptide (>3-fold increase). This suggests that the role of the diacylglycerol is to facilitate transportation of the peptide across the cell membrane and the cytokine/chemokine expression that is mediated by pDAG arises from the peptide alone.

Accordingly, it is to be appreciated that the present invention provides a new immune modulating compound originally isolated from caprine serum as 1-peptidyl-2-arachidonoyl-3-stearoyl glyceride (pDAG), the peptide portion from which was found to be 100% homologous to a unique region of TRPC-1. After synthesis of pDAG, according to the present invention it has been demonstrated that both pDAG and the transfected peptide moiety of pDAG can modulate the signaling in human fibroblasts with an EC₉₉ in the range of 1-10 nM. IL-6 and IL-8 are important innate immune cytokines that are not only expressed by fibroblasts and other non-immune cells, but are also expressed by activated immune cells. IL-6 and TGF are required for the differentiation of naïve mouse CD4+ T-lymphocytes into TH17 cells. See Mangan, P. R.; Harrington, L. E.; O'Quinn, D. B.; Helms, W. S.; Bullard, D. C.; Elson, C. O.; Hatton, R. D.; Wahl, S. M.; Schoeb, T. R.; and Weaver, C. T. Nature 2006, 441, 231-234. More recently it has been shown that human TH17 cell differentiation is mediated by IL-1, IL-6 and IL-23. See Wilson, N. J.; Boniface, K.; Chan, J. R.; McKenzie, B. S.; Blumenschein, W. S.; Mattson, J. D.; Basham, B. Smith, K. Chen, T.; Morel, F.; Lecron, J.-L.; Kastelein, R. A.; Cua, D. J.; McClanahan, t. K.; Bowman, E. P.; and Malefyt, R. deW. Nat. Immunol. 2007, 8, 950-957.

Data produced in the development of the present invention suggests that the peptide in pDAG may activate the host innate immune response against pathogen infection or other cellular injury. Furthermore, pDAG and the transfected peptide moiety of pDAG has been found to stimulate the increased expression of the chemokines, MCP-1 and MIP-1α in fibroblasts and the function of these proteins are to recruit leukocytes and macrophages to the region. This suggests a role for pDAG and the peptide moiety of pDAG as a damage associated molecular patterns in the host response to pathogen infection or other cell injury to clear pathogens and dead or injured cells. Whereas pDAG was isolated from normal caprine serum, we have been unable to detect pDAG in normal caprine, equine, non-human primate (cynamologous monkey) or human plasma, but have been able to detect pDAG in the serum of each at the estimated concentration of 50 ng/mL. It is believed that pDAG is derived from platelets in the clotting process and is not present in normal plasma. However, pDAG may be present in the plasma of sepsis patients or patients with certain chronic idiopathic diseases. We are currently evaluating this hypothesis as well.

### EXAMPLES

General Experimental Procedures. Chromatography of the serum fractions was performed on an Ultimate 3000 HPLC (Dionex, Sunnyvale, Calif.), with a Zorbax C₈ 1×150 mm column (Agilent, Santa Clara, Calif.). Mass spectrometry of the natural product (pDAG) was performed on a LTQ linear ion trap mass spectrometer (ThermoFisher Scientific, Pittsburgh, Pa.). Separation was achieved using a Paradigm MS4 HPLC (Michrom Bioresources) and HTS-PAL autosampler (LEAP Technologies) with a 20 L sample loop and a 1×50 mm MAGIC C₁₈ reversed phase column (Michrom Bioresources) and an in-line UV detector monitoring the column effluent at 280 nm (ABI 759A with 2.4 L microbore flow cell). Matrix-assisted laser desorption/ionization time-of-flight (MALDI-TOF) mass spectra of purified natural product were acquired in the positive-reflectron mode using a Kratos AXIMA-CFR mass spectrometer (Manchester, UK). The instrument was equipped with a 337 nm nitrogen laser, a 20-kV extraction voltage, and time-delayed extraction. Saturated dihydroxybenzoic acid in 50% acetonitrile and 10% tribasic ammonium citrate (9:1) served as the matrix. Automated Edman degradation sequence analysis was performed using a Procise Protein Sequencer (Applied Biosystems, Inc.). Peptide synthesis was performed on an AAPPTEC 348 Sigma peptide synthesizer (Advanced Automated Peptide Protein Technologies, Inc., Louisville, Ky.). ESIMS-MS analysis of the synthetic product (pDAG) was performed using a Thermo-Finnegan Surveyor HPLC-LCQ DECA Ion Trap LCMS-MS in the +ve mode (ThermoFisher Scientific, Pittsburgh, Pa.).

Animal Material. Normal, sterile, endotoxin-free, goat serum was collected from a closed, disease-free herd (EquitechBio, Inc., Kerrville, Tex.) and shipped frozen in 500 mL bottles for extraction.

Extraction and Chromatographic Isolation. Serum (500 mL) was thawed at room temperature and dialyzed in 10 kDa molecular weight cut-off tubing (SnakeSkin®, ThermoFisher Scientific) against deionized water for 24 h with stirring in a cold room. The dialysate was frozen and lyophilized and the solid residue extracted with 0.1 mL of methanol/chloroform (2:1, v/v) for each 1 mg of solid residue. The un-dissolved residue was collected by centrifugation and the methanol/chloroform soluble material was recovered after evaporation in vacuo. Bioassays indicated that the methanol:chloroform insoluble residue contained pDAG (Table 4, below).

TABLE 4
IL-8 ELISA Bioassay Results of Serum Fractions^{a}

| | untreated | | | |
|---|---|---|---|---|
| fraction | control | 1 mg/mL | 100 g/mL | 10 g/mL |
| Dialysate | 0.099 | 3.50 | 3.49 | 0.469 |
| MeOH/CHCl₃ Soluble | 0.084 | 0.111 | 0.147 | 0.126 |
| MeOH/CHCl₃ Insoluble | 0.114 | 3.80 | 3.75 | 2.14 |

^{a}Data are presented as the means of duplicate absorbance (450 nm) measurements.

This fraction was dissolved in 10 mL of deionized water and dialyzed in a 7 KDa molecular weight cut-off dialysis cassette (Slide-a-Lyzer®, Thermo Scientific) against deionized water at 4° C. with stirring (200 rpm) for 24 hours. This second dialysate was frozen and lyophilized. The solid residue was extracted with 0.1 mL methanol/chloroform (2:1, v/v) for each 1.0 mg of solid. The methanol/chloroform soluble fraction was separated from the undissolved solids by centrifugation and evaporated in vacuo. Biological assay showed that compound pDAG was again in the methanol/chloroform insoluble fraction (Table 5, below).

TABLE 5
Serum Fractions Induced mRNA Expression in Human Fibroblasts^{a}

| | | | methanol/ | methanol/ |
|---|---|---|---|---|
| | untreated | | chloroform | chloroform |
| mRNA | control | retentate | soluble | insoluble |
| IL-6 | 52.3 | 271.1 | 66.4 | 423.5 |
| IL-8 | 4.4 | 166.5 | 6.9 | 337.5 |
| MCP1 | 56.9 | 197.1 | 86.1 | 254.3 |
| MIP-1 | 82.4 | 404.6 | 91.9 | 414.2 |

^{a}Data are the means of duplicate absorbance (450 nm) measurements.

A sample (200 L) in water was chromatographically analyzed. The solvent gradient was 5-65% solvent A (5% acetonitrile+0.1% TFA) to solvent B (90% acetonitrile+0.1% TFA) over 60 min., followed by a 5-min. wash with 90% solvent B, at a flow rate of 50 L/min. Detection was at 214 nm and 280 nm. Fractions were manually collected every minute for bioassay.

N-Terminal Sequence Analysis of the Natural Product (pDAG). Phenylisothiocyanate was reacted with the terminal amino group of the peptide to form a phenylthiocarbamoyl derivative. Then, under mildly acidic conditions, the terminal amino acid was removed to produce a phenylthiohydantoin (PTH) derivative of the amino acid, and generate a free amino group on the next residue in the peptide sequence. The PTH-AA was quantitatively identified by HPLC by comparison to the retention time of the known standard reference PTH-AA. This process was then repeated to identify the next amino acid. At the end of each cycle during sequencing, results of the chromatography were automatically collected in data files. The amount (in pmol) of each amino acid detected in each cycle and the difference in yield for each amino acid in each cycle compared to the previous cycle were calculated.

ESIMS-MS of the Natural Product (pDAG). HPLC purified pDAG (0.7 mg) was dissolved in 0.5 mL of 0.1% formic acid in water and 10 L were loaded onto the column through the sample loop. The sample was eluted using a binary gradient where solvent A was 98% H₂O and 2% acetonitrile with 0.1% formic acid and 0.01% trifluoroacetic acid and solvent B 10% H₂O and 90% acetonitrile with 0.1% formic acid and 0.01% trifluoroacetic acid. The gradient was formed from the initial conditions of 2% B to 50% B over 20 min. and then washed with 100% B for 5 min.

MALDITOFMS of the Natural Product (pDAG) The sample of pDAG was dissolved in chloroform/methanol (4:1) and 0.8 μL deposited onto the sample plate with an equal volume of the matrix solution. The sample mixtures were dried at room temperature prior to mass analysis. Hexa-acylated lipid A 1,4’-bisphosphate from wild type E. coli (Sigma Aldrich, St. Louis, Mo.) served as an external standard for calibration.

Hydrolysis and Derivatization of the Natural Product (pDAG). Native pDAG is labile to acid hydrolysis. Therefore, 100 g of native pDAG was subjected to mild acid hydrolysis conditions (1 N HCl in phosphate buffered saline) for 18 hours at 25° C. The post-reaction buffer salts were removed using a C₁₈ spin column (ThermoFisher Scientific) and the hydrolysis products were eluted from the column using 50% CH₃CN/0.1% TFA. The final product was brought to dryness by evaporation in vacuo at 4° C. The hydrolysis products were derivatized using the N-terminal sulfonation reagent, 4-sulfophenylisothiocyanate, and analyzed in situ by MALDITOF in an -cyano-4-hydroxycinnamic acid matrix using the method described by Wang. See Wang, D.; Kalb, S. R.; Cotter, R. J. Rapid Commun. Mass Spectrom. 2004, 18, 96-102.

4-sulfophenylisothiocyanate was dissolved in 20 mM sodium bicarbonate (pH 9.5) to a final concentration of 10 μg/μL. A 50-fold excess of the 4-sulfophenylisothiocyanate solution (80 μL) was added to 4 μL of pDAG (4 μg/μL). The sulfonation reaction was incubated at 55° C. for 30 min. and the reaction was quenched by addition of 5% trifluoroacetic acid. The sample was loaded onto a micropipette tip (C₄ ZipTip; Millipore), washed with 3×10 μL of 0.1% trifluoroacetic acid, and eluted with 50% CH₃CN/0.1% trifluoroacetic acid. The analysis of the sulfonated hydrolysis products of pDAG were conducted on the AXIMA-CFR MALDITOFMS in the positive-reflectron mode.

1-Peptidyl-2-arachidonoyl-3-stearoyl glyceride (pDAG). The peptide in pDAG was synthesized by a standard solid phase synthesis protocol with extended HBTU coupling on H-Isoleucine-2-Chlorotrityl resin. The fully protected peptide sequence conjugated to the resin [Ala-Leu-Tyr(But)-Asp(OBut)-Lys(Boc)-Gly-Tyr(But)-Thr(But)-Ser(But)-Lys(Boc)-Glu(OBut)-Gln(Trt)-Lys(Boc)-Asp(OBut)-Cys(Trt)-Val-Gly-Ile-RESIN] was recovered after washing with dichloromethane. The peptide conjugated resin was acetylated at the N-terminal alanine of the peptide by the addition of 10% acetic anhydride in N,N-diisopropylethylamine (20%) and N,N-dimethylacetamide (70%). After two hours at room temperature the resin was filtered, washed successively with N,N-dimethylacetamide and dichloromethane, and lyophilized. The acetylated and fully protected peptide was cleaved from the resin using 20 mL of a 1:4 solution of 1,1,1,3,3,3-hexafluoro-2-propanol in dichloromethane. After two hours at room temperature, the resin was filtered and washed with 2 mL of the cleavage solution. The filtrate was evaporated in vacuo using a rotary evaporator. A sample was analyzed by mass spectrometry to confirm the expected mass at m/z 3286.

1-Stearoyl-2-arachidonoyl-sn-glycerol (Sigma Aldrich, St. Louis, Mo.) was esterified to the C-terminal isoleucine carboxyl of the fully protected acetylated peptide using the dicyclohexylcarbodiimide/dimethylaminopyridine (DCC/DMAP) coupling reaction. 1-stearoyl-2-arachidonoyl-sn-glycerol (5 mg) was dissolved in 2 mL of dichlormethane and mixed with 2 equivalents of the fully protected acetylated peptide dissolved in 2 mL of dichloromethane and 1 equivalent of DCC/DMAP also dissolved in 2 mL of dichloromethane. The reaction was allowed to proceed overnight at room temperature. The reaction mixture was dried in vacuo and the protecting groups were removed in situ by the addition of 8 mL of the deprotection solution (2.5% 1,2-ethandiol, 94% trifluoroacetic acid, 0.1% triisopropylsilane, and 2.5% water). After a two hour incubation period at room temperature in the presence of the deprotecting solution the reaction mixture was filtered and the filtrate was evaporated in vacuo.

Purification of the crude product was accomplished by reversed-phase preparative scale HPLC using a Jupiter® Proteo column (Phenomenex, Inc., Torrance, Calif.) and a binary mobile phase gradient (Solvent A: 0.1% trifluoroacetic acid in water Solvent B: 0.1% trifluoroacetic acid in acetonitrile) formed from 5% to 95% Solvent B over 20 minutes (4.5% per minute) at a flow rate of 1 mL/minute and the effluent was continuously monitored at 220 nm. The synthetic product (pDAG) eluted at 22.9 minutes under these conditions. The crude product (63 mg) was dissolved in 4 mL of acetonitrile and 2 mL of water and loaded onto the preparative scale column. The synthetic product was collected in 22.5 mL of the mobile phase (95% acetonitrile/0.1% trifluoroacetic acid). The effluent was evaporated in vacuo. The final mass of synthetic product (pDAG) was 2.5 mg (4% yield) with a purity as determined by HPLC analysis to be 98.9%.

ESIMS-MS of Synthetic pDAG. In one experiment the mobile phase was A [1% 2-propanol, 0.03% trifluoroacetic acid in H₂O] and B [1% 2-propanol (containing 2 mmol NH₄OAc in 9:1 2-propanol:H₂O) 0.03% trifluoroacetic acid in CH₃CN]. In a second experiment the mobile phase was A [0.1% formic acid in H₂O] and B [CH₃OH]. The column was post-treated with 0.1M LiOH by syringe pump at 5 μL/min. The mobile phase gradient was formed from 5% B-80% B (10 min.), 80% B-95% B (13.0-27.0 min.), 95% B (27.0-35.0 min.), 95% B-5% B (35.0-36.0 min.) and 5% B (36.0-41.0 min). The column was a Zorbax XDB-C₈, 3.5 μm, 2.1×50 mm.

mRNA Expression Experiments. Normal primary human fibroblasts obtained from the Coriell Institute (Camden, N.J.) were cultured in 70 mm dishes in DMEM supplemented with 10% FBS and 1× penicillin/streptomycin at 37° C. and 5% CO₂. Human fibroblasts were incubated in the presence of the serum fractions, compound pDAG, the peptide moiety of pDAG, or diacylglycerol. In the initial experiments we examined the differences between fibroblasts treated with the various serum fractions, synthetic pDAG (the peptide moiety from pDAG or diacylglycerol), fibroblasts treated with LPS as a positive control, and fibroblasts without treatment. The culture medium was stored at −80° C. for ELISA of the excreted cytokine proteins (see below). Total RNA from fibroblasts was extracted using the RNeasy Mini kit (Qiagen, Valencia, Calif.) according to the manufacturer's protocol. To verify the expression of transcripts; 2.0 μg of total RNA was reverse-transcribed using Super-script-III reverse transcriptase (Invitrogen, Carlsbad Calif.), according to the manufacturer's protocol. Transcripts were quantified using SYBR Green PCR amplification (Perkin Elmer, Waltham Mass.). Primers employed in these studies are outlined in Table 6, below. All transcripts were normalized to β-actin. Briefly, 0.4 μg/μL of cDNA was tested with 10 μM of each specific primer and 1× SYBR Green in a total volume of 20 uL.

TABLE 6
Primer Sequences for Real-Time PCR

| g [symbol] | forward primer | reverse primer |
|---|---|---|
| I [symbol] | 5′-GGCCCTAAACAGATGAAGTGC [symbol] | 5′-GGCCTGCCTGAAGCCC-3′ |
| I [symbol] | 5′-TCCTTCTCCACAAGCGCC-3′ | 5′-AAGGCAGCAGGCAACAAC-3′ |
| I [symbol] | 5′-CTGCAGCTCTGTGTGAAGGTGC [symbol] | 5′-GGTGGAAAGGTTTGGAGTATGTCTTTA [symbol] |
| I [symbol] | 5′-CTGTAGAGATAATGCACCCCGG [symbol] | 5′-GTTCTCACAGGAGAGAGTTGAAATTTTC [symbol] 3′ |
| I [symbol] | 5′-GGAACACTCTGTGGAGCTCCAT [symbol] | 5′-CACCTATAAACACTCCAGGATCAGTC [symbol] |
| [symbol] | 5′-GCTCGCTCAGCCAGATGCAATC [symbol] | 5′-GGACACTTGCTGCTGGTGATTCTTC-3′ |
| [symbol] | 5′-GAGAACTTCTTAAAGGGCTGCCA [symbol] 3′ | 5′-CTGCCCCTGCCTAGATTCTCATACC-3′ |
| | 5′-TTGCCGACAGGATGCAGAA-3′ | 5′-GCCGATCCACACGGAGTACTT-3′ |

[symbol]

indicates data missing or illegible when filed

ELISA for IL-8 Protein. Human IL-8 was measured by ELISA according to the manufacturer's specifications (eBioscience San Diego, Calif.) in the supernatants of fibroblasts cultured in complete DMEM with varying amounts of pDAG. Briefly, high affinity binding plates were coated with anti-human IL-8 overnight at 4° C. Wells were washed with Wash Buffer five times at 1 min. each wash and blocked with Assay Diluent for 60 min. at room temp. Samples were added to the wells along with diluted standards and incubated for 2 h. Wells were washed and detection antibody was applied for 30 min., wells washed again, an Avidin-HRP antibody applied for 30 min., unbound antibody removed by washing as described, and tetramethylbenzidine substrate added for 15 min. The reaction was halted with Stop Solution and the plate read at 450 nm with wavelength subtraction at 570 nm.

Lipofection Studies. Cells were cultured so that at the time of transfection they were 50-80% confluent. Five microliters of the stock peptide moiety from pDAG was diluted in Opti-MEM Reduced Serum Medium to give a final concentration of 5 μg/mL. PLUS reagent (0.5 μL) and lipofectamine (1 μL) were then added to the peptide Opti-MEM solution, vortexed and incubated at room temperature for 30 min. Five hundred microliters of complete DMEM was added to the fibroblasts and 0.6 μL of the peptide/lipofectamine was added and allowed to incubate overnight. RNA was extracted and processed as described above.

Bioassays. The fractions generated from dialysis (methanol/chloroform soluble, methanol/chloroform insoluble, dialysis retentate, and the fractions collected from reversed phase HPLC) were taken to dryness by lyophilization. The freeze-dried solids were weighed and dissolved in the appropriate volume of deionized water to give a final concentration of 1 mg/mL and assayed for mRNA expression (IL-6, IL-8, MIP1α, and MIP1β) in human fibroblasts.

To demonstrate the physiological effect of the administration of pDAG, pDAG produced in accordance with the present invention as described herein was administered in various amounts to cows and dogs, the former to ascertain the effect of pDAG on the number of white blood cells remaining after administration and the latter to ascertain the effect of pDAG on the survival rate of dogs receiving doses of pDAG. In each case pDAG was effective in mitigation of established bacterial and viral infections; in the first case reducing the number of white blood cells by nearly threefold, and in the second case over doubling the survival rate of the pDAG treated dogs by comparison with that of untreated dogs. pDAG produced in accordance with the invention was also administered to rabbits, and a significant adjuvant effect was found to occur in terms of IgM production when pDAG was administered with other drugs.

Further experiments have shown that pDAG produced in accordance with the invention when administered in dosages of 25 ng/ml and 50 ng/ml is capable of the reduction of C. pneumonia load in THP-1 cells, and that it is also capable of reduction of HBV viral load in hepatocytes at dosages of from 25 to 400 ng/ml. The pDAG produced in accordance with the invention is believed to induce fibroblast IL-6 expression similar to that of the natural product, and to induce fibroblast IL-8 mRNA and protein expression, to increase MCP-1 expression and to induce MIP-1alpha. It is presently believed that pDAG signals through the inflammasome in cells, and that IL-6 is mediated by caspase-1, which is only activated by inflammasome-mediated signaling. pDAG in accordance with this invention was also found to induce IL-18 and IL-33, as well as IL-1alpha and IL-1beta.

It is to be appreciated that many additional modifications and variations, that will be apparent to those skilled in the art in view of the disclosure herein, may be made in the specific embodiments of the invention as described herein, and that all such modifications are fully within the scope of the present invention. Accordingly, it is to be appreciated that while certain aspects of the preferred embodiments of the present invention have been described, it is not intended that the invention be limited to such embodiments. Various modifications may be made thereto without departing from the spirit of the present invention, the full scope of which is delineated solely in the following claims.

What is claimed is:

1. A compound having the formula:

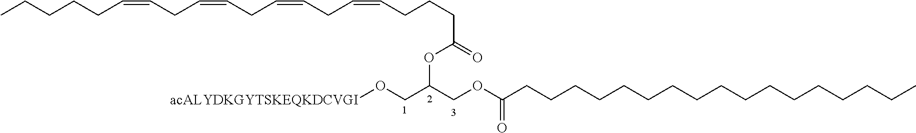

2. The compound of claim 1, wherein said compound an active chemical entity in the caprine serum fraction.

3. The compound of claim 1, wherein said compound has innate immune modulating activity as an endogenous damage associated molecular pattern.

4. 1-peptidyl-2-arachidonoyl-3-stearoyl glyceride.

5. A method for inducing inflammatory cytokine and chemokine production and release from human fibroblasts, which method comprises administering to said fibroblasts a physiologically effective dose of a compound having the formula:

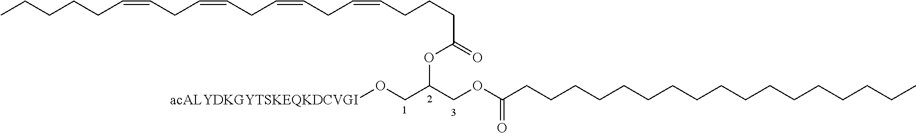

6. A method for the treatment and mitigation of bacterial and viral infections in a subject, which method comprises administering to the subject a physiologically effective dose of a compound having the formula: [chemical structure]
